(19) United States
(12) Patent Application Publication
(10) Pub. No.: US 2007/0068534 A1
(43) Pub. Date: Mar. 29, 2007

(54) Apparatus for maximizing airflow patency

(75) Inventors: Dennis R. Bailey, Englewood, CO (US); Dennis F. Creedon, Sandwich, MA (US); Lewis H. Marten, Westwood, MA (US)

Correspondence Address: FROMMER LAWRENCE & HAUG, 745 FIFTH AVENUE- 10TH FL., NEW YORK, NY, 10151, US

(73) Assignee: E. Benson Hood Laboratories

(21) Appl. No.: 11/234,032

(22) Filed: Sep. 23, 2005

(51) Int. Cl. A61F 5/56

(52) U.S. Cl. 128/848

(57) ABSTRACT

The present invention relates to an apparatus for the maximization of airflow patency. A bite jig is disclosed that is adaptable to work in conjunction with a mouthpiece. This combination can then be connected to an acoustic pharyngometer. The system allows for the simultaneous, or near simultaneous, measuring of airflow capacity and mandibular adjustment.

### FIELD OF THE INVENTION

The present invention relates to mandibular advancement devices, or oral appliance devices, used to minimize airflow restrictions during sleep. These devices commonly treat orthodontic conditions, bruxism, snoring, Obstructive Sleep Apnea (“OSA”), other sleep breathing disorders and temporomandibular joint disorders. More specifically, the present invention relates to a combination bite jig, mouthpiece and acoustic pharyngometer capable of quickly and objectively maximizing airflow patency.

### BACKGROUND OF THE INVENTION

Certain sleep breathing disorders, including OSA and snoring, occur as a result of airway obstruction. Mandibular advancement devices force the lower jaw forward and downward. The repositioning of the mandible pulls the tongue forward and thereby decreases upper airway obstruction.

Mandibular advancement devices come in numerous forms. Moldable mouthpieces, similar to dental protective devices worn by athletes, exist to retain the lower jaw in a forward position during sleep. One such example is set forth in U.S. Pat. No. 5,462,066. This and similar moldable mouthpieces offer a one-time custom fit and retain the lower jaw in a static position during sleep.

Mandibular advancement devices utilizing moldable mouthpieces, however, suffer from several problems. In addition to taking time to create, such devices are often tough to create properly, especially for a first time user. Often the resulting mold is too loose. This can be due to, for example, a failure to apply the proper suction or a failure to heat the device to the proper molding temperature during creation. Although the user can sometimes correct such an improper mold creation by a second heating and forming attempt, after two attempts such devices are often incapable of creating a proper fit. Even if properly created, these devices lack adjustability.

User adjustable mandibular advancement devices also exist. U.S. Pat. No. 5,570,704, for example, discloses a user adjustable oral cavity device designed to reduce snoring and other sleep breathing disorders. The device includes a means by which the user can adjust the degree of separation between the maxilla and mandible. The device also includes a means by which the user can adjust the degree to which the mandible is moved forward.

Other mandibular advancement devices allow for lateral excursion of the mandible relative to the upper jaw. U.S. Pat. No. 5,868,138 discloses such a device. The lateral movement relieves discomfort to the tempromandibular joint, related muscles and ligaments exacerbated by laterally fixed devices for users that have a tendency to grind their teeth during sleep.

Some mandibular advancement devices, such as the one disclosed by U.S. Pat. No. 5,611,355, are both moldable and adjustable. In the '355 patent, the upper and lower members mold to the users teeth. Once molded, these members fasten together using an adjustable fastening mechanism, such as hook-and-loop fasteners, permitting the lower member to be gradually moved forward over a period of days or weeks.

U.S. Pat. No. 6,055,986 discloses a mandibular advancement device incorporating and combining many of the features of the aforementioned devices. Specifically, the '986 patent discloses an oral apparatus including moldable upper and lower members adjustably attached. The attachment means allows for both forward and lateral freedom of movement of the mandible.

Other oral appliances decrease upper airway obstruction by repositioning the user's tongue. Like mandibular advancement devices, these oral appliances help to normalize breathing during sleep thus relieving snoring and OSA. An example is seen in U.S. Pat. No. 6,675,804. There, a semi-spherical multi-lobed structure positions the user's tongue. In addition to this tongue positioning structure, this oral appliance includes a flanged lip plate and a dome-shaped outer shield.

Several problems exit when using these devices. The degree of adjustability in these devices is limited. Additionally, even when these devices are adjustable, they are often cumbersome to adjust and contain numerous separate interrelated moving parts. As such, they are subject to breakage. Furthermore, optimization of settings remains difficult if not impossible. For prior art devices, the mandibular advancement or other oral appliance device settings are determined by combining trial and error with patient feedback. The user or physician is left to guess at the optimal settings. The inability to precisely optimize the settings, such that the particular oral appliance device results in the largest upper airway opening possible, leaves users with, at best, incomplete or partial relief.

Pharyngometers and other wavetube-based devices are known in the art. U.S. Pat. No. 5,666,960, incorporated herein by reference, is an example. Acoustic Pharyngometers graphically depict airway patency by using acoustic signaling processing technology. An acoustic signal is generated by a probe position in the mouth and subsequently recorded by microphones. An acoustic reflectometer plots a cross-sectional area of the airway as a function of distance from the airway opening by measuring the amplitudes and arrival times of the acoustic signals. These devices typically comprise a mouthpiece, wavetube, speaker, microphone, acoustic device, filter strips, CPU, printer, monitor, P.C. board, software and source code. Typically these devices have various electronic components affixed to the wavetube. These devices provide fast, objective analysis of airway obstruction and give the physician concrete data with which to make adjustments in the patient's oral appliance or mandibular advancement device.

While Pharyngometers and other wavetube-based devices provide objective data on a patient's airway obstruction, these devices are not designed to be used in connection with a particular oral appliance or mandibular advancement device. As a result, the objectively obtained data does not translate into an optimum setting for a particular oral appliance.

The present invention is designed to overcome the aforementioned problems. Specifically, the present invention is directed to an oral appliance device, of durable construction, capable of precise mandibular adjustment while adapted to being used in conjunction with a mouthpiece and acoustic Pharyngometer. This arrangement allows for the simultaneous, or near simultaneous, airflow capacity measurement and mandibular adjustment.

### SUMMARY OF THE INVENTION

It is therefore an object of the invention to provide an apparatus for maximizing airway patency comprising a bite jig; a mouthpiece; and, an acoustic pharyngometer.

Further object of the invention will be achieved by a the specific bite jig configuration including a handle, a center section adapted to receive user's front teeth, an arch support member and at least one arch, disposed between the arch support member and the user's teeth.

A further object of the invention is provide a mouthpiece adaptable to be connected to an acoustic Pharyngometer including an arched flange and a lumen, wherein the lumen includes ridges adapted to receive a connection to an acoustic Pharyngometer.

Still further objects of the invention will be achieved by a tongue guide affixed to the bite jig and by connecting an adjustable tongue guide to the bite jig.

Still further objects of the invention will be achieved by providing a plurality of protuberances on the bite jig handle.

Other objects of the invention will be achieved by providing a center section of the bite jig that includes at least one upper cutout, adapted to receive at least one upper tooth of the user.

Other objects of the invention will be achieved by providing a center section of the bite jig that includes at least one lower cutout, adapted to receive at least one lower tooth.

### BRIEF DESCRIPTION OF THE DRAWINGS

Further objects and advantages of the invention will become apparent from the following description and claims and from the accompanying drawings wherein:

FIG. 1 is a top view of one embodiment of the bite jig of the present invention shown without a tongue guide;

FIG. 2 is a side view of the bite jig shown in FIG. 1;

FIG. 3 is a top view of one embodiment of the bite jig of the present invention shown with a tongue guide;

FIG. 4 is a side view of the bite jig shown in FIG. 3;

FIG. 5 is a top view of one embodiment of the mouthpiece of the present invention;

FIG. 6 is a side view of the mouthpiece shown in FIG. 5;

FIG. 7 is a top view of one embodiment of the assembled bite jig and mouthpiece of the present invention shown without a tongue guide;

FIG. 8 is a side view of the assembled bite jig and mouthpiece shown in FIG. 7.

### DETAILED DESCRIPTION

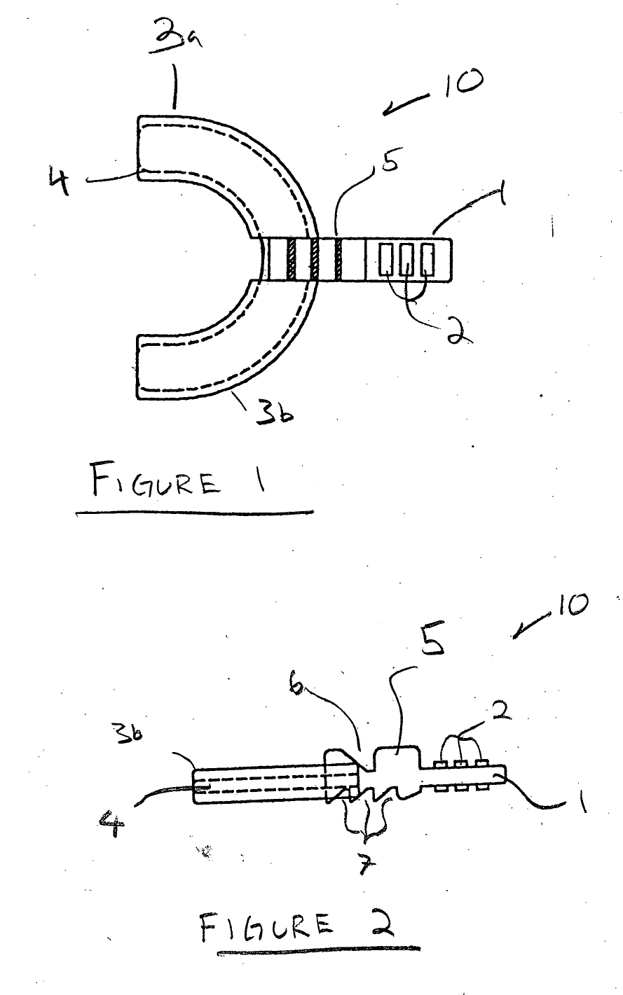

Turning now more particularly to the drawings, FIG. 1 shows an embodiment of the bite jig of the present invention without a tongue guide. The bite jig operates to adjust the mandible relative to the maxilla. Bite jig 10 includes a handle 1, center section 5, arch support piece 4, and arches 3. Handle 1 aids the introduction of the bite jig 10 into the user's mouth. The handle includes protuberances 2 to provide for better grip and finder control during insertion.

In this embodiment of the present invention there are a total of two arches. These arches, 3a and 3b, are shown in FIG. 1. As seen in FIG. 1, arch 3a protrudes to the left of (upwardly from) the base of handle 1. Arch 3b protrudes to the right of (downwardly from) the base of handle 1. The arches are adapted to receive at least some of the user's teeth upon insertion of the bite jig into the oral cavity. Preferably, the arches are made of a soft material, such as silicone, in order to comfortably receive a number of possible configurations of teeth. The soft arches also provide bearing points as an aid to maintain stability of the jaw during acoustic testing. In this embodiment of the present invention, the arches are constructed as a single unit with a center opening or slit such that arch supporting piece 4 may be inserted into, and thus support, the arches as shown in FIG. 1. In this way the arches are easily removable and replaceable. In other embodiments, the arches could be affixed directly to the support piece 4 by adhesive, fasteners or other means, whether easily removable or not. In such an embodiment, the two arches could be separated into four different arches, two upper and two lower or the arches could be configured as a unitary upper arch and a separate unitary lower arch. It should also be noted that the arches of the present invention may, in different embodiments, be a variety of thicknesses, whether uniform or not, in order to best fit a particular patient's anatomy.

FIG. 2 shows a side or profile view of the bite jig shown in FIG. 1. Arch 3b is shown with arch support 4 (dotted lines) inserted into the center opening of arch 3b. It can be seen that support piece 4 is attached to the bite jig center section 5, which is in turn connected to handle 1. Center section 5 has a series of cutouts. Upper cutout 6, located on the top of center section 5, holds the position of the user's upper teeth. When the user bites down on the bite jig the upper front teeth or upper incisors enter into upper cutout 6. This cutout operates to hold the position of the upper teeth and aids in the setting of various positions of the mandible.

Center section 5 also includes a plurality of lower cutouts 7. When the user bites down on the bite jig the lower front teeth or lower incisors enter into and will be held by any one of the lower cutouts 7. As such, these cutouts operate to hold the position of the lower mandible. The lower cutouts 7 are evenly spaced to guide the movement of the lower jaw forward. The lower cutouts 7 are positioned to allow for normal closure of jaws and a gradual advancement of lower jaw.

In this embodiment of the present invention handle 1, center section 5, and arch support piece 4 are a single unitary construction manufactured out of a semi-rigid material. This construction and material allow for a secure positioning of the lower jaw. The thickness of this unitary construction can vary. For example, the degree of oral cavity opening will be dictated by the thickness of center section 5 and support piece 4. As such, in different embodiments, the bite jig is provided in various thicknesses at center section 5 and along support piece 4, where the user's teeth are positioned. The present invention envisages numerous types of thickness variation. A substantially uniform thickness throughout section 5 and support piece 4 could be incrementally increased in successive embodiments. And in other embodiments the thickness could vary throughout section 5 and support piece 4, forming, for example, a wedge shape. These different embodiments allow for adjustment of the user's bite registration and further allows for testing to aid in setting of various changes in incisal openings.

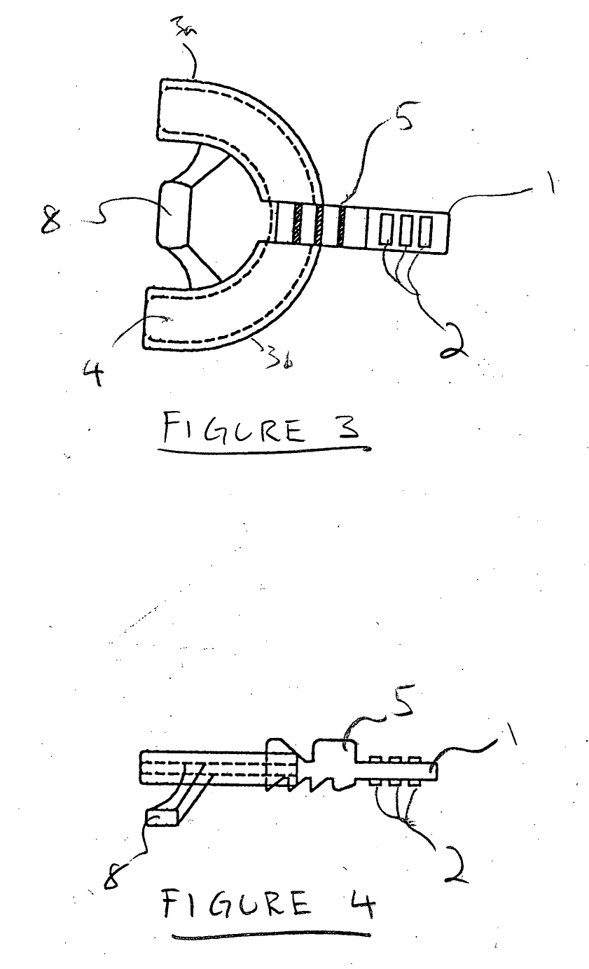

Turning now to FIGS. 3 and 4, another embodiment of the present invention includes a tongue guide 8. Tongue guide 8 causes the tongue to lie low in the mouth and move forward. In addition, the tongue guide allows for the unimpeded transmission of sound during acoustic testing. The tongue guide also encourages a restriction of the movement of various tongue muscles. As shown in FIGS. 3 and 4, the tongue guide is affixed to support piece 4. In other embodiments the tongue guide is adjustable. In these embodiments tongue guide 8 is removably attached to support piece 4 through the use of, for example, notches in support piece 4 and corresponding attachment members at the ends of tongue guide 8. In this way, the tongue guide can be adjusted to provide for maximum airflow and still further allow for the unimpeded transmission of sound during acoustic testing.

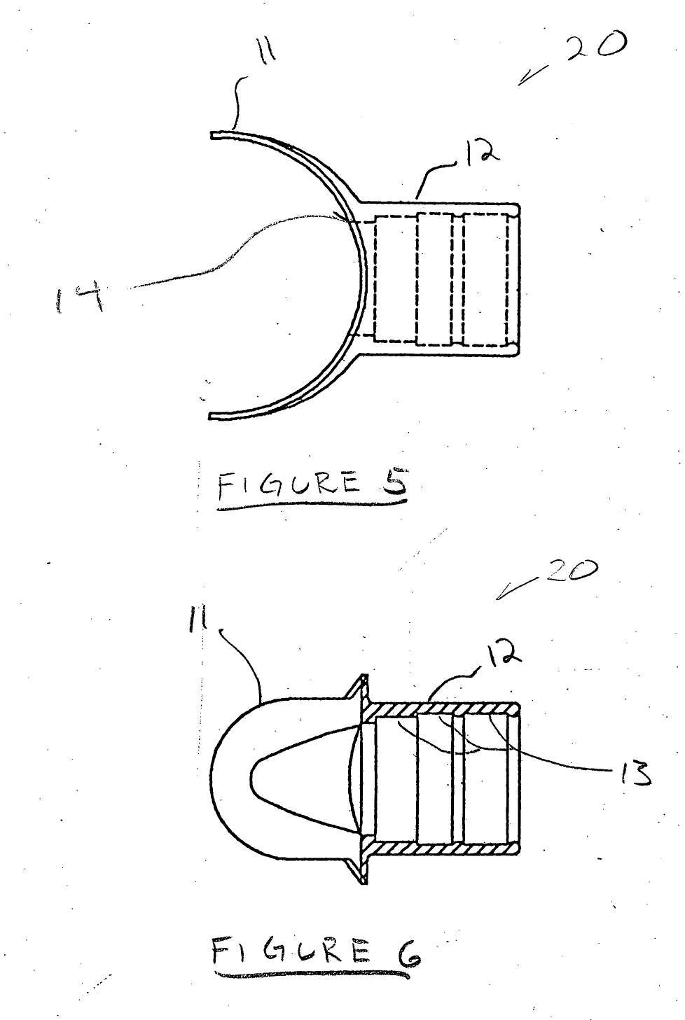

The bite jig is designed for use in conjunction with a mouthpiece or acoustic sealing flange. An embodiment of such a mouthpiece can be seen in FIG. 5 (top view) and FIG. 6 (side view). There, mouthpiece 20 is shown including flange section 11 and lumen 12. Flange section 11 includes arch 14. Mouthpiece 20 is manufactured of a soft and flexible material to allow for a positive acoustic seal against the user's gums. Although the mouthpiece 20 is designed and sized to fit adolescents and adults without any need for adjustment, the soft material may be trimmed to fit the user's mouth in extreme cases if necessary.

Mouthpiece 20 also includes internal ridges 13 formed on the inside of lumen 12. In other embodiments the ridges are on the outside of lumen 12. These ridges aid in connecting the mouthpiece to a Pharyngometer. Specifically, the mouthpiece 20 is connected to the Pharyngometer wave tube. The ridges allow for ease of coupling and uncoupling the mouthpiece to the Pharyngometer wave tube, while maintaining a stable connection during the testing process.

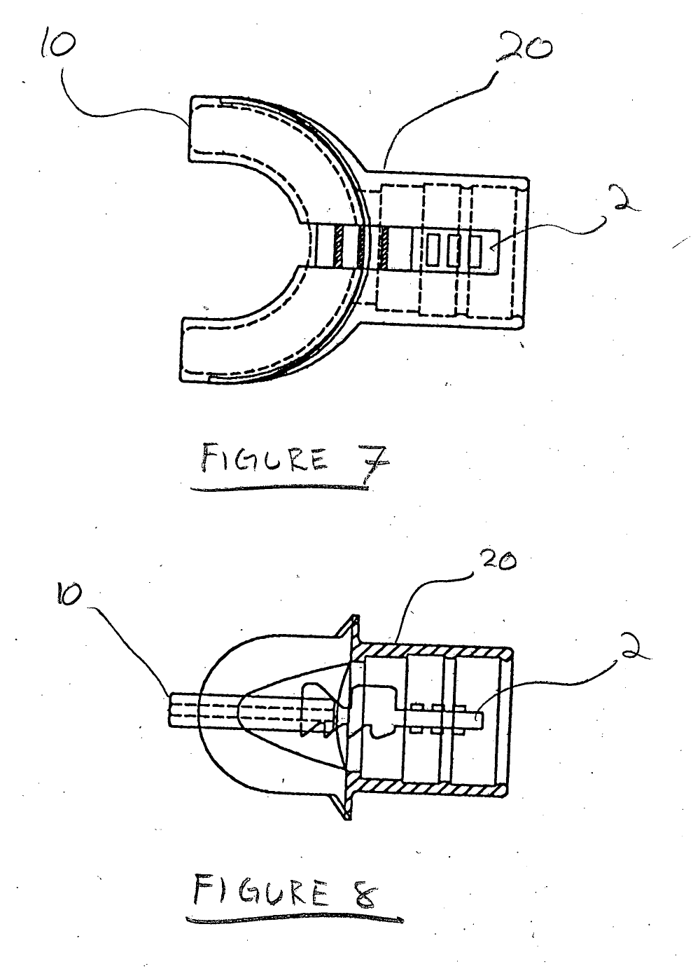

FIGS. 7 and 8 show the bite jig 10 and the mouthpiece 20. The bite jig 10 is used in conjunction with the mouthpiece, which provides for minimal leakage of acoustic reflection during testing. The mouthpiece 20 is shaped to allow the bite jig arches to fit inside the flange section arch 14, thus requiring minimal space and optimal fit inside the user's mouth.

In operation, the present invention allows for the near simultaneous measuring of airflow capacity and mandibular advancement device adjustment. The physician first places the bit-jig into the users mouth. The user bites down on the bite jig. Here, the mandible is adjusted to an initial position. The mouthpiece is then placed into the users mouth. As seen most clearly in FIGS. 7 and 8, the bite jig handle 2 is inserted into the mouthpiece lumen 12. Then mouthpiece flange section 11 is placed between the users lips and gums. As noted above, mouthpiece 20 is manufactured of a soft and flexible material to allow for a positive acoustic seal against the user's gums. At this point, the bite jig handle 2 is suspended in approximately the center of lumen 12 by the combination of the user's biting action and the placement of the mouthpiece flange section 11 between the users lips and gums. Next, a Pharyngometer wave tube is connected to lumen 12.

With this apparatus, the physician is then able to test the user's airway patency. After receiving objective data from the Pharyngometer, the physician can easily and quickly adjust the advancement of the user's mandible without removing or disconnecting the Pharyngometer. This allows for the precise and simultaneous, or near simultaneous, measuring of airflow patency and mandibular adjustment.

What is claimed is:

1. An apparatus for maximizing airway patency comprising:
a bite jig;
a mouthpiece adapted to receive said bite jig; and,
an acoustic Pharyngometer in fluid communication with said mouthpiece.

2. The apparatus of claim 1, wherein the bite jig comprises:
a handle;
a center section integrally connected to said handle, adapted to receive a user's front teeth;
an arch support member integrally connected to said center section;
at least one arch, disposed between the arch support member and the user's teeth.

3. The apparatus of claim 1, wherein the mouthpiece comprises:
an arched flange; and
a lumen integrally connected to said arched flange;
wherein the lumen includes ridges adapted to receive a connection to an acoustic Pharyngometer wave tube.

4. The apparatus of claim 2, wherein the mouthpiece comprises:
an arched flange; and
a lumen integrally connected to said arched flange;
wherein the lumen includes ridges adapted to receive a connection to an acoustic Pharyngometer wave tube.

5. The apparatus of claim 1 further comprising a tongue guide affixed to the bite jig.

6. The apparatus of claim 1 further comprising an adjustable tongue guide connected to said bite jig.

7. A bite jig comprising:
a handle;
a center section integrally connected to said handle, adapted to receive a user's front teeth;
an arch support member integrally connected to said center section;
at least one arch, disposed between the arch support member and the user's teeth.

8. The bite jig of claim 7 wherein the handle includes a plurality of protuberances.

9. The bite jig of claim 7 wherein the center section includes at least one upper cutout, adapted to receive at least one upper tooth.

10. The bite jig of claim 7 wherein the center section includes at least one lower cutout, adapted to receive at least one lower tooth.

11. The bite jig of claim 9 wherein the center section includes at least one lower cutout, adapted to receive at least one lower tooth.

12. The bite jig of claim 7 further comprising a tongue guide affixed to said arch support member.

13. The bite jig of claim 7 further comprising an adjustable tongue guide connected to said arch support member.

14. The bite jig of claim 11 further comprising a tongue guide affixed to said arch support member.

15. The bite jig of claim 11 further comprising an adjustable tongue guide connected to said arch support member.

16. A mouthpiece comprising:
an arched flange; and
a lumen integrally connected to said arched flange;
wherein the lumen includes ridges adapted to receive a connection to an acoustic Pharyngometer wave tube.
